(19) United States
(12) Patent Application Publication
(10) Pub. No.: US 2025/0113846 A1
(43) Pub. Date: Apr. 10, 2025

(54) CORN PROTEIN CONCENTRATE AND METHODS OF MANUFACTURING SAME

(71) Applicant: Cargill, Incorporated, Wayzata, MN (US)

(72) Inventors: Yumin CHEN, Gurnee, IL (US); Eugene Max PETERS, SR., Kettering, OH (US); Michael A. PORTER, Maple Grove, MN (US); Craig A. WILSON, Sidney, OH (US); Hadi Nayef YEHIA, Beavercreek, OH (US); Guo-Hua ZHENG, Centerville, OH (US)

(73) Assignee: Cargill, Incorporated, Wayzata, MN (US)

(21) Appl. No.: 18/987,147

(22) Filed: Dec. 19, 2024

Related U.S. Application Data
(63) Continuation of application No. 16/086,765, filed on Sep. 20, 2018.
(60) Provisional application No. 62/312,867, filed on Mar. 24, 2016.

(51) Int. Cl. A23L 5/20; A23J 1/00; A23J 3/14; A23L 7/10; C07K 14/415

(52) U.S. Cl. CPC A23L 5/23; A23J 1/006; A23J 3/14; A23L 5/20; A23L 7/198; C07K 14/415

(57) ABSTRACT

Described herein is a corn protein concentrate comprising 55%-80% corn protein on a dry basis, an a* color value between about 0 and 4, and a b* color value between about 15 and 3, and less than about 2% oil on a dry basis; and a method of manufacturing the same.

### CROSS-REFERENCE TO RELATED APPLICATIONS

This application is continuation of Ser. No. 16/086,765, filed Sep. 20, 2018, which is a national phase application of PCT/US2017/023999, filed Mar. 24, 2017, which claims the benefit of U.S. Provisional Patent Application No. 62/312,867, filed Mar. 24, 2016, each of which is hereby incorporated by reference in its entirety.

### TECHNICAL FIELD

This disclosure relates to corn protein concentrate and methods of manufacturing the same.

### BACKGROUND

For over 100 years, corn wet milling has been used to separate corn kernels into products such as starch, protein, fiber and oil. Corn wet milling is a two stage process that includes a steeping process to soften the corn kernel to facilitate the next wet milling process step that results in purified starch and different co-products such as oil, fiber, and protein. Further corn processing methods are now being investigated to further purify the protein co-product for incorporation into food-grade products, specifically.

### SUMMARY

Described herein is a corn protein concentrate comprising 55%-80% corn protein on a dry basis, an a* color value between about 0 and 4, and a b* color value between about 15 and 3, and less than about 2% oil on a dry basis.

Further described herein is a method of producing a corn protein concentrate, comprising providing a corn gluten meal, washing the corn gluten meal with a solvent comprising water and a water-miscible organic solvent to obtain a corn protein concentrate comprising 55%-80% corn protein on a dry basis, an a* color value between about 0 and 4, and a b* color value between about 15 and 3, and less than about 2% oil on a dry basis.

### BRIEF DESCRIPTION OF DRAWINGS

FIGS. 1-4 illustrate viscosity properties of the various corn protein concentrates described in the examples herein.

FIG. 5 illustrates the appearance of the various corn protein concentrates described in the examples herein compared against a corn protein isolate product.

FIG. 6 illustrates the appearance of dried corn gluten meal after unmolding.

### DETAILED DESCRIPTION

The process of producing a corn protein concentrate starts with a corn gluten meal typically comprising at least about 55 wt % protein on a dry basis (note that all reference to percentages herein are weight percentages unless stated otherwise). In most aspects, the starch in the corn gluten meal remains intact and does not undergo a destarching enzymatic hydrolysis process. Similarly, protein structure in the corn gluten remains intact, in most aspects, and does not undergo a denaturation/coagulation process under heat conditions.

The corn gluten meal may then be washed with a water-miscible solvent. In aspects of the present invention, the water-miscible solvent may be an ethanol-containing or isopropanol-containing solvent in concentrations ranging from about 85 wt % to about 99.5 wt %, preferably 85 wt % to about 98 wt % (ethanol or isopropanol), and more preferably in concentrations ranging from 85 wt % to about 95 wt % (ethanol or isopropanol).

A series of solvent washing steps may be performed to remove non-protein, non-starch components. In preferred aspects, there are no more than six solvent washing steps.

Surprisingly, the solvent washes described herein were found to remove many non-protein components (pigments, organic acids, oils, sulfites, etc.) from the starting corn gluten meal, thus enhancing the recovery of the corn protein concentrate as described in more detail below.

In certain aspects, both corn gluten meal and the solvent are introduced in a mixing tank and vigorously mixed for about 15 minutes. To reduce the amount of non-protein, non-starch components contained in the mixture, the mixture goes through an extraction and filtration step. Such extraction may be carried out using a batch stir tank, continuous stir tank reactor or by percolation or immersion extraction. In certain aspects, filtration is carried out using a Buchner funnel to filter out the non-protein, non-starch component-containing solvent and maintain the protein stream. It shall also be understood, however, that while filtration is used in an aspect of this process, other separation techniques such as drainage, percolation, centrifugation or decanting may be utilized to achieve the separation of the non-protein, non-starch component-containing solvent from the protein-containing stream.

The protein-containing stream undergoes another solvent washing, extraction and filtration step and, in preferred aspects, yet another solvent washing, extraction and filtration step therefore achieving three solvent washing steps. This solvent washing step is repeated once more before the protein-containing stream is dried in a desolventizer before recovering the corn protein concentrate.

A goal of the solvent washing process described above is to concentrate the corn protein-starch composition by removal of other non-protein components. Notably, the process described herein produces a corn protein concentrate product comprising 55-80 wt % corn protein on a dry basis (db), and in preferred aspects a corn protein concentrate product comprising 55-75 wt % (db) corn protein.

Another goal of the presently described process is the removal of residual oils, carbohydrates, organic acids, and pigment. The process described herein decreases the oil content so that it makes up less than 2 wt % (db) of the corn protein concentrate, more preferably less than 1.5 wt % (db) and more preferably less than 1 wt % (db).

Furthermore, the process described herein produces a corn protein concentrate wherein the total insoluble carbohydrate concentration ranging from about 15-18 g/kg, with insoluble carbohydrates, having a series of glucose polymers comprising three glucose units linked with alpha 1,4-glycosidic linkages (maltotriose or DP3) and greater than three glucose units (DP4), comprising at least about 75% of the total insoluble carbohydrate concentration. In the same way that polar solvents favor the extraction of carbohydrates, they also favor the extraction of organic acids. As described herein, organic acids include citric acid, succinic acid, lactate, glycerol, acetate, and propionic acid. Steeping of corn gives rise to a variety of organic acids and some remain in the starting corn protein material used as the raw material for this process. The residual total organic acid concentration in the corn protein concentrate after solvent extraction is about 3.0 g/kg or less.

The starting corn gluten meal may be yellowish-orange in color because most of the corn pigments (luteins, zeaxanthins, cryptoxanthins, and carotenes) concentrate into the protein stream. This color is undesirable for most food-grade applications. Accordingly, the solvent washing step described herein eliminates a substantial amount of the color and provides a corn protein concentrate product having an “a*” color value between about 0 and 4 (and more preferably between 0 and 2), a “b*” color value between about 15 and 35 (and more preferably between 15 and 30), and an “L*” color value ranging between about 70 and 90 (and more preferably between 80 and 90).

There are also circumstances where the primary benefits of the corn protein concentrate are functional-particularly with respect to interactions with water. For example, a benefit of adding protein ingredients to processed meats is enhanced water holding through the cooking process and in such applications, the starch provides a potential benefit. Accordingly, the resulting corn protein concentrate also comprises starch levels ranging from 13 wt % to 23 wt % (db), and more preferably from 13 wt % to 16 wt % (db). Further, there's a desire to reduce the amount of free sulfite for food labeling purposes. The corn protein concentrate described herein has a free sulfite concentration less than 100 ppm.

Even more specifically, the presence of starch in the corn protein concentrate of the present invention provides desirable gelation properties in certain food applications, for example processed meat applications. The corn protein concentrate described herein has a gel strength ranging from about 0.15 to 0.20 N, and more preferably a gel strength ranging from about 0.15 to 0.20 N.

### EXAMPLES

### Materials & Methods

Raw materials for these experiments were collected using a pilot-scale vacuum drum filter to collect and dewater corn gluten meal (CGM) slurry from standard corn wet mill processing. The slurry was collected on the drum filter, rinsed with 1% H₂O₂ at a wash ratio of approximately 8% and after further draining was collected in plastic bags and frozen. Material was held frozen until use. Frozen CGM was thawed at room temperature before use-generally in the day preceding the work in some combination of room temperature and refrigerator conditions. Corn starch (Argo brand) was acquired from a local grocery.

For starch analysis, the method consists of boiling the CPC sample with aqueous calcium chloride solution to solubilize the starch and then measuring the optical activity of the solution with a polarimeter (derived from Method G-28 of the Corn Refiners Association Standard Analytical Methods).

### Example 1: Bench Scale Development

A series of experiments were conducted to explore the effect of different solvent regimes on extraction in three steps.

Sample CPC070815-1: 200 g of corn gluten meal cake (58.4% moisture, from Cargill Corn Milling, Wahpeton) is suspended in 1000 g of absolute EtOH. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g 90% w/w EtOH and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight.

Sample CPC070815-2: 200 g of corn gluten meal cake (58.4% moisture, from Cargill Corn Milling, Wahpeton, ND) is suspended in 1000 g of isopropanol. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g 90% w/w isopropanol and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight.

Sample CPC070815-3: 200 g of corn gluten meal cake (58.4% moisture, from Cargill Corn Milling, Wahpeton, ND) is suspended in 1000 g of solvent comprising 80% w/w ethyl acetate, 20% w/w EtOH. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g 72% w/w ethyl acetate, 18% w/w ethanol, 10% w/w water and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight.

Sample CPC070815-4: 90 g of freeze dried CGM (2.93% moisture by moisture balance, from Cargill Corn Milling, Wahpeton, ND) is extracted in 1000 g of hexane. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g hexane and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight. The cake is conspicuously yellow-orange and the extraction solution is relatively pale yellow.

Sample CPC070815-5: 90 g of freeze dried CGM (2.93% moisture by moisture balance, from Cargill Corn Milling, Wahpeton, ND) is extracted in 1000 g of absolute EtOH. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g absolute EtOH and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight. The cake is conspicuously yellow-orange and the extraction solution was relatively pale yellow. These samples filtered very fast compared to the usual extractions in 90% EtOH.

Sample CPC070815-6: 200 g of wet CGM cake (58.4% moisture, from Cargill Corn Milling, Wahpeton, ND) is extracted in 1000 g of absolute EtOH. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g 90% w/w EtOH in water and left stirring for 15 minutes. The entire process is repeated one more time for a total of three washes. The final cake is drained for approx. 2 minutes beyond the point when the surface solvent disappeared. The cake is spread out in a pie plate and desolventized in a hood initially then in a vacuum oven at about 65° C. overnight. The cake is conspicuously yellow-orange and the extraction solution is relatively pale yellow.

Sample CPC070815-7: 200 g of wet CGM cake (58.4% moisture, from Cargill Corn Milling, Wahpeton, ND) is extracted in 1000 g of absolute EtOH. After intense mixing with an immersion blender the suspension is left stirring for 15 minutes. The suspension is poured into an 18.5 cm Buchner funnel lined with VWR417 filter paper and drained under vacuum. When the drip rate is about 1/sec, the cake is collected and re-suspended in 1000 g 90% w/w EtOH in water and left stirring for 15 minutes. The entire process is repeated one more time in this manner. The cake is then washed two times for ten minutes with 1000 g 90% w/w EtOH in water. The final cake is drained for approx. 2 minutes beyond the point when the surface solvent disappeared. The cake is spread out in a pie plate and desolventized in a hood briefly then in a vacuum oven at about 65° C. overnight. The cake is conspicuously yellow-orange and the extraction solution is relatively pale yellow.

Before extraction, CGM is about 67% protein (db) and 4-6% oil (db). All of the solvents tested decreased the oil content by 80% or more (Table 1). Protein concentrations are equal or somewhat higher after extraction. The color metric L* increases considerably when wet CGM cake is used as starting material, but less so when freeze-dried material is used. The color measures a* and b* are decreased in almost all cases, with the greatest effect apparent with 90% isopropanol. Some protein may have been lost during extraction as some corn proteins are solvent soluble, especially in the absence of prior heat treatment. In contrast, starch is insoluble in these solvents and is concentrated through the process.

TABLE 1
Composition of products extracted at bench scale

| | Solvent | | | | | | |
|---|---|---|---|---|---|---|---|
| | (non-aqueous | Protein | Oil | Starch | | | |
| | fraction, w/w) | (% db) | (% db) | (% db) | L* | a* | b* |
| Corn Gluten | Pre-extraction, | 67.3 | 5.75 | 12.9 | 86.22 | 5.56 | 37.81 |
| Meal | freeze-dried CGM | | | | | | |
| Corn Gluten | Pre-extraction, | 66.65 | 3.94 | 15.0 | 63.69 | 16.33 | 53.1 |
| Meal | wet CGM | | | | | | |
| CPC070815-1 | 90% EtOH | 71.46 | 0.72 | 15.8 | 82.11 | 1.18 | 19.8 |
| CPC070815-2 | 90% Isopropanol | 70.34 | 0.53 | 15.8 | 88.58 | 0.03 | 16.08 |
| CPC070815-3 | 72% w/w ethyl | 70.25 | 0.69 | 14.3 | 83.82 | 0.73 | 18.25 |
| | acetate, | | | | | | |
| | 18% w/w ethanol | | | | | | |
| CPC070815-4 | 100% hexane | 72.07 | 0.46 | 15.9 | 86.31 | 3.72 | 30.95 |
| CPC070815-5 | 100% EtOH | 68.89 | 0.16 | 15.6 | 86.35 | 2.61 | 26.54 |
| CPC070815-6 | 90% EtOH | 68.60 | 0.12 | 13.9 | 74.84 | 3.31 | 25.58 |

### Example 2: Pilot Scale Development

Two pilot trials were conducted to test initial conditions for processing of CGM. The behavior of CGM in the extractor and desolventizer created some issues with stickiness and solvent removal, but are attributed to the presence of starch and partial protein solubilization. Subsequent trials were conducted to produce samples for functional testing. The various operating conditions are shown in Tables 2 and 3.

TABLE 2
Extractor conditions in Pilot trials used to test initial conditions and
compositional impact.

| | CPC P- | CPC-P- |
|---|---|---|
| Primary Production set points | 041615-1 | 072715-02 |
| Solvent Feed Rate (lbs./min) | 0.97 | 0.89 |
| Feed rate (wet lbs./min) | Not measured | 0.06 |
| Input EtOH density (g/mL) | Not measured | 0.81033 |
| Input [EtOH] % w/w | 50.74 | 62.43 |
| Extractor Temperature (° C.) | 0.97 | 64.51 |
| Desolventizer Jacket pressure (psig) | ~70 | Not measured |
| Desolventizer Discharge Temp (° C.) | Not measured | 260 (15%) |
| Desolv. Rotor Speed (rpm) | Not measured | 338.91 |
| Desolventizer Vacuum pressure | 79.07 | 64.51 |
| (mbar) | | |

TABLE 3
Extractor Conditions in Pilot Trials used to produce functional prototypes.

| Primary Production set | CPC-P-012116- | CPC-P-012216- |
|---|---|---|
| points | 119 (“CPC119”) | 120 (“CPC120”) |
| Solvent Feed Rate | 0.98 | 0.98 |
| (lbs./min) | | |
| Feed rate | 0.06 | 0.06 |
| (wet lbs./min) | | |
| Input EtOH density (g/mL) | 0.8107 | 0.7970 |
| Input [EtOH] % w/w | 92.5 | 97.5 |
| Extractor Temperature (° C.) | 55.65 | 59.49 |
| Desolventizer Jacket pressure | 85.22 | 83.93 |
| (psig) | | |
| Desolventizer Discharge | 89.5 | 110.59 |
| Temp (° C.) | | |
| Desolv. Rotor Speed (rpm) | 260 (15%) | 260 (15%) |
| Desolventizer Vacuum | 331.4 | 281.16 |
| pressure (mbar) | | |

The first two attempts to produce a CPC at pilot scale are primarily concerned with understanding processing issues. The first attempt resulted in a higher protein, higher oil composition than the second attempt (Table 4), but demonstrates that the process could be used to significantly decrease the oil content and pigment of CGM (compare to data in Table 5).

TABLE 4
The basic composition of initial pilot produced CPC samples.

| | Protein | Oil | Starch | | | |
|---|---|---|---|---|---|---|
| | (% db) | (% db) | (% db) | L* | a* | b* |
| CPC P-041615-1 | 77.7 | 1.37 | 12.4 | 86.2 | 3.5 | 26.3 |
| CPC-P-072715-02 | 63.28 | 0.51 | 22.4 | 79.66 | 3.49 | 24.09 |

In the subsequent pilot trials, the protein concentrations were lower and starch concentrations were higher, partly due to higher initial starch concentrations (Table 5). Without being bound by any theories, one explanation may be that extraction conditions dissolved sufficient protein (mainly alpha-zein) to reduce the final concentration because starch was not extracted in a proportional manner. Oil concentration is decreased more than 95%. The input EtOH concentration used is higher in the production of CPC120 than CPC119, but there is about the same final protein concentration. In a complementary manner, the starch concentration is higher in the finished product than the starting material.

TABLE 5
The basic composition of pilot produced CPC samples.

| | | Protein | Oil | Starch | Sulfite |
|---|---|---|---|---|---|
| Lot# | % LOD | (% bd) | (% bd) | (% db) | (mg/kg) |
| Corn Gluten Meal | 61.58 | 62.9 | 4.87 | 17.7 | 66 |
| CPC-P-012116- | 4.35 | 54.9 | 0.11 | 34.3 | 89 |
| 119 | | | | | |
| CPC-P-012216- | 2.78 | 57.5 | 0.21 | 31.5 | 83 |
| 120 | | | | | |

Table 6 shows that extraction decreased monosaccharides, had almost no impact on di- and trisaccharides, while “concentrating” higher oligomers. Extraction also removed 90% or more of the lactic acid (Table 7). It is unclear whether there was any change in the succinic acid or citric acid concentrations.

TABLE 6
The carbohydrate composition of pilot produced CPC samples

| | Carbohydrates (g/kg) on db | | | | | |
|---|---|---|---|---|---|---|
| | DP3+ | DP3 | MT | Dx | Fx | Total |
| CGM-10-15-2015 | 0.9 | 0.8 | 0.8 | 8.4 | 3.5 | 14.4 |
| CPC-P-012116-119 | 14.7 | 0.7 | 0.5 | 1.2 | 0.5 | 17.7 |
| CPC-P-012216-120 | 12.9 | 0.8 | 0.6 | 1.4 | 0.9 | 16.4 |

TABLE 7
The organic acid composition of pilot produced CPC samples

| | Organic Acids (g/Kg) on db | | | | | | |
|---|---|---|---|---|---|---|---|
| | Citric | Succinic | Lactate | Glycerol | Acetate | Propionic* | Total |
| CGM-10-15-2015 | 1.3 | 0.0 | 6.7 | 0.0 | 0.2 | 0.4 | 8.6 |
| CPC-P-012116-119 | 1.1 | 0.2 | 0.3 | 0.0 | 0.0 | 0.0 | 1.6 |
| CPC-P-012216-120 | 1.1 | 0.4 | 0.7 | 0.0 | 0.0 | 0.2 | 2.5 |

Removal of pigments during pilot extraction results in an overall lighter product (Table 8) with significant declines in the residual pigments contributing to yellow (a*) and red (b*). The higher EtOH concentration used with CPC120 seems to result in a less intensely colored product across all three measures.

TABLE 8
The color measures of pilot produced CPC samples

| | Color | | |
|---|---|---|---|
| | L* | a* | b* |
| CGM-10-15-2015 | 64.4 | 15.2 | 55.1 |
| CPC-P-012116-119 | 85.5 | 1.1 | 20.5 |
| CPC-P-012216-120 | 89.9 | 0.5 | 17.2 |

One of the hypothesis behind development of a lower protein variant is that the starch present from the CGM will have a strong affinity for water and can be used to hold water through a cooking cycle. The samples from the earliest attempts were never tested for functionality, but samples CPC119 and CPC120 are suitable for functional testing. Two measures of functional behavior were particularly notable: viscosity and gelation.

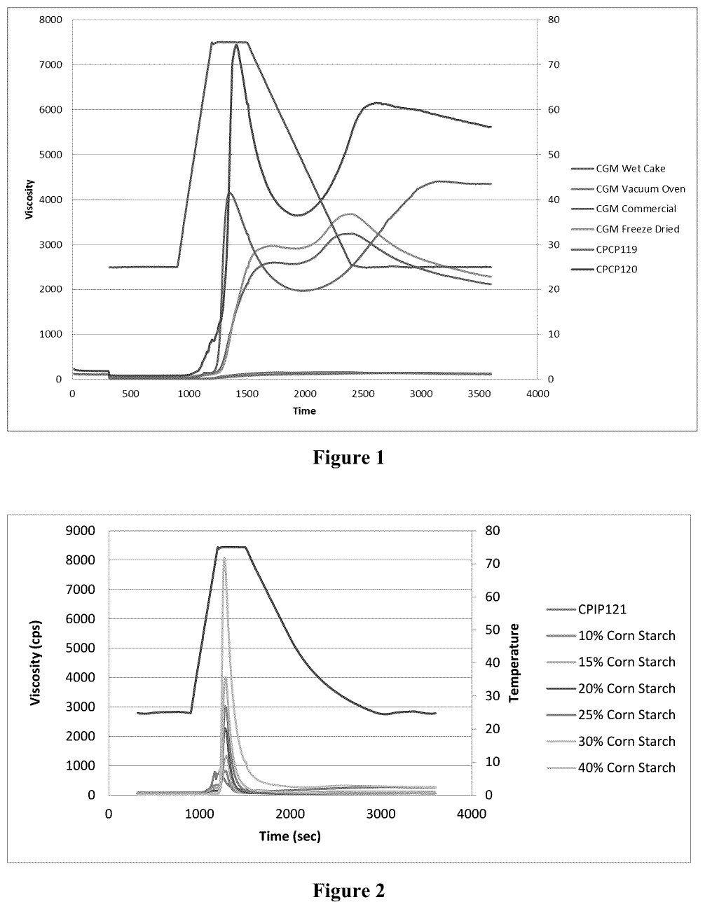

The two pilot corn protein concentrate samples have very high viscosities compared to an equal concentration of corn protein isolate (CPI) (Table 9 and FIG. 1). Table 9 shows the viscosity measures for CPC119 and CPC120. For comparison, CPIP121 represents a high protein version (protein>85%). CGM Cake is corn gluten meal collected at Cargill Corn Milling, Wahpeton, ND and frozen until shortly before use. This sample is thawed on the bench top and diluted to the desired concentration. CGM Vacuum Oven represents the same sample of frozen CGM after drying at 70° C. in a vacuum oven. CGM (Blair Production) is a commercial sample of CGM (Cargill Corn Milling, Blair, NE). The higher temperatures experienced by the product after drying may gelatinize the starch and modify the protein behavior in subsequent testing. CGM FD represents a sample of the same CGM (Cargill Corn Milling, Wahpeton, ND) that was freeze dried to eliminate additional prior heat exposure.

Dried CGM (whether in the vacuum oven or production dryer) has a relatively low viscosity at each measuring point and shows relatively little structure in the response (FIG. 1). CGM cake that had never been dried generates more viscosity, particularly during the latter stages of high temperature treatment and cooling. Freeze-dried CGM created a viscosity profile similar to the wet cake, showing that the difference in behavior is more likely to be a consequence of heating than drying per se. CPC119 and CPC120 have much higher peak viscosity and much higher final viscosity. There is a clear distinction between the behavior of the two CPC samples and the CPI sample.

TABLE 9
The viscosity of 20% w/w dispersions of various intermediate protein
prototypes through a heating-cooling cycle. Data for CPC119, CPC120
and CPI121 are means of duplicate analyses.

| | | | Viscosity | |
|---|---|---|---|---|
| | Viscosity | Viscosity | 25° C. After | Max |
| Sample | Initial 25° C. | 75° C. | Heating | Viscosity |
| CGM Cake | 65 | 2129 | 2117 | 3245 |
| CGM Vacuum | 7 | 124 | 110 | 161 |
| Oven | | | | |
| CGM (Blair | 8 | 81 | 129 | 146 |
| Production) | | | | |
| CGM FD | 51 | 2487 | 2284 | 3680 |
| CPC119 | 27 | 3225 | 4347 | 4408 |
| CPC120 | 85 | 6125 | 5619 | 7433 |
| CPI121 | 99 | 199 | 281 | 1005 |

Two features of the viscosity profiles for CPC samples should be noted. First, they reach a peak viscosity later in the heating cycle than the more pure protein variant. Second, upon cooling they initially lose some viscosity, but then regain all or more of the lost viscosity to finish near the peak viscosity. Samples CPC119 and CPC120 differ in peak viscosity. This seems like the most dramatic consequence of the differing solvent compositions used in extraction.

This might be the expected consequence of replacing some of the protein with starch, but FIG. 2 shows that something else may be happening. While peak viscosity rises as the starch component increases (FIG. 3), the final viscosity does not. In addition, the peak viscosity of the starch alone (at 6% inclusion-equivalent to replacing 30% of the protein with starch) is not at all near the level of the protein-starch composition. This is further emphasized in the comparison of the viscosity at 25° C., 75° C., at 25° C. after cooling (FIG. 4). The comparisons make clear that the co-processed protein-starch combination has different functionality than the independently processed and then mixed combination. It might be speculated that the aqueous EtOH induced an interaction between the starch and protein with a subsequent effect on the maintenance of viscosity after cooling.

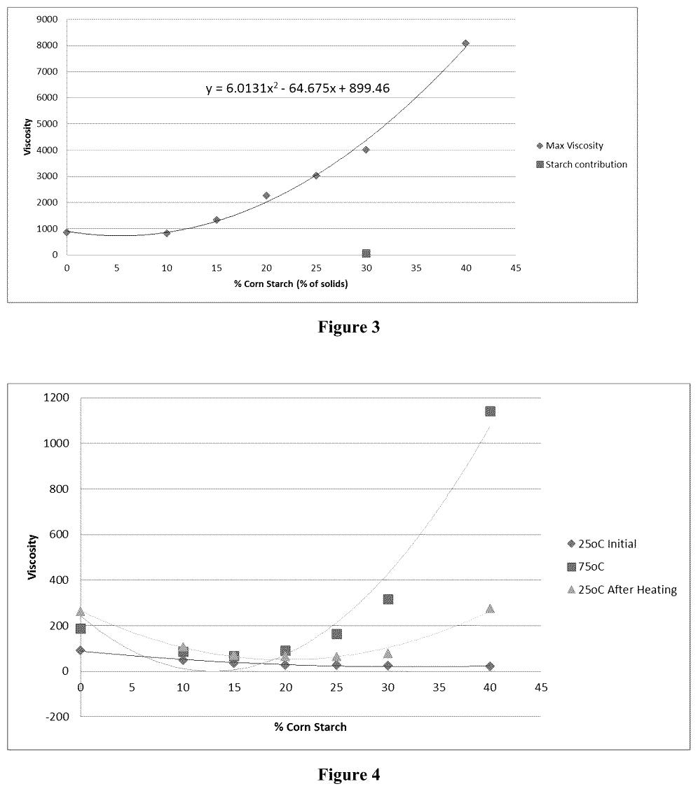

The viscosity results suggest that samples CPC119 and CPC120 should form good gels and this is what was observed. FIG. 5 shows the comparison between the appearance of the CPC 119 (bottom), CPC120 corn protein concentrate (top, left) and a corn protein isolate (CPI121) (top, right). The CPC forms a firm gel with a defined edge and shape. This is a high quality gel. The strength of the gel was measured to be 18.4 g or 0.18 N (Table 10). The CPI sample (CPI121) was a thickened dispersion with no measurable gel strength. Though CPC119 and CPC120 are similar, the extraction in higher EtOH apparently created a firmer gel that was apparent both visually and instrumentally.

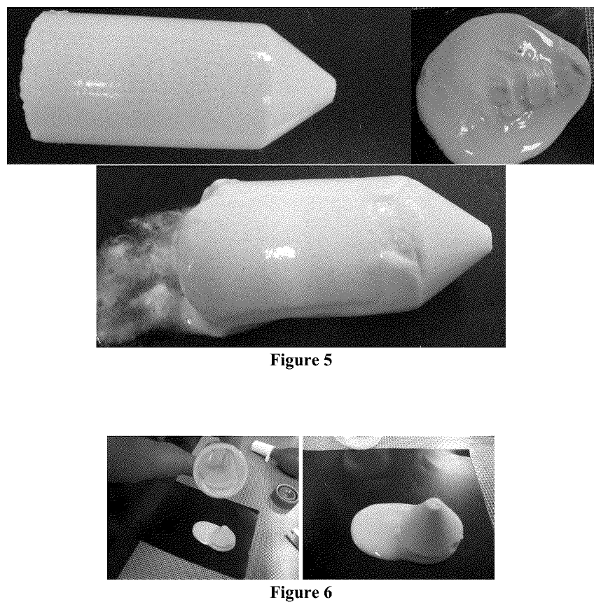

CGM that was dried in a vacuum oven, ground and then tested in the gel method formed a solid at the tube's tip where the particulates had settled (FIG. 6). The gel strength of the tip portion was 18.2 g (0.18 N), but this is clearly not the sort of uniform comprehensive gel formed by the CPC sample. So despite a similar composition, the sample of vacuum oven-dried CGM cake could not create gel behavior like the CPC.

TABLE 10
Gel hardness and visual score. The visual score runs from 1 (=low
quality) to 5 (high quality).

| Sample | Gel hardness (N) | Visual Score |
|---|---|---|
| CPI-P-012116-119 | 0.119 | 4 |
| CPI-P-012216-120 | 0.181 | 5 |
| CPIP121 | <0.098 | 1 |

### Example 3: CPC Production from Corn Gluten Meal

Corn gluten cake is collected on a rotary drum vacuum filter with rinsing. The destarched slurry is fed to the drum at 1.2 gal/min at a density of about 1.016 g/mL. The rinse water supplemented with hydrogen peroxide to a concentration of 0.31% w/w active hydrogen peroxide is applied at 0.12 gal/min. Upon completion of the vacuum dewatering, the treated cake is frozen until further use.

10 kg of peroxide-treated, corn gluten cake with 60-65% moisture is processed through a dual rotor crusher with a 0.125-inch screen to generate a uniformly sized particle for homogeneous extraction. The cake is fed to a Crown Iron Works Model IV immersion extractor using a drag conveyor dropping through a crossover screw and then a delumper (for a better understanding, an illustration of the Crown Iron Works Model IV immersion extractor may be found on the crowniron.com website) into the extractor. The extractor includes a series of inclined drag conveyors arranged so that the lower end of the conveyor is submerged in the extraction solvent and the upper end was above the solvent. The conveyor carried the solids forward such that the material is initially submerged in solvent and then the material emerged from the solvent and excess solvent drained back into the solvent stream. At the end of the conveyor, the solids dropped onto another conveyor with a similar arrangement. The model IV extractor has six extraction stages. Fresh solvent is introduced at the discharge end and flowed towards the inlet end and is ultimately discharged at a point preceding the solids introduction.

After the final solvent contact, the solids are conveyed up a long section to allow more extensive draining before falling into a crossover screw for transport to desolventizing. The solvent is fed into the system at 0.445 kg/min and the solids are introduced at 0.027 kg/min (based on a calibrated volumetric feeder) and the solvent is maintained at 56° C. by in situ heat exchangers. Total solvent to solids ratio is about 16 and total contact time is about 60 minutes. The water of the extraction system is introduced through a combination of carryover water from the input material and water in the fresh solvent. The composition of the feed solvent to contact the extracted destarched corn gluten is approximately 92.2% ethanol and 7.8% water. Consequently, the composition of the solvent varied across the extractor, but the final solvent concentration is about 92% ethanol.

Desolventizing occurred in a Bepex Solidaire dryer operated with a surface temperature of about 155-160° C. and an absolute pressure from about 270-330 millibar (with a target of about 300 millibar).

The resulting corn protein concentrate product is about 54.9% protein (dry basis). Further, the oil is less than 0.5% on a dry basis, the product color, as measured using the Hunter colorimeter, has “L*” color equal to 85.5, “a*” color equal to 1.1 and “b*” color equal to 20.5. The free sulfite is 89 mg/kg (dry basis).

### Example 4: CPC Production from Corn Gluten Meal

Corn gluten cake is collected on a rotary drum vacuum filter with rinsing. The destarched slurry is fed to the drum at 1.2 gal/min at a density of about 1.016 g/mL. The rinse water supplemented with hydrogen peroxide to a concentration of with 1% w/w is applied at 0.12 gal/min. Upon completion of the vacuum dewatering, the treated cake is frozen until further use.

10 kg of peroxide-treated, corn gluten cake with 60-65% % moisture is processed through a dual rotor crusher with a 0.125-inch screen to generate a uniformly sized particle for homogeneous extraction. The cake is fed to a Crown Iron Works Model IV immersion extractor using a drag conveyor dropping through a crossover screw and then a delumper (for a better understanding, an illustration of the Crown Iron Works Model IV immersion extractor may be found on the crowniron.com website) into the extractor. The extractor includes a series of inclined drag conveyors arranged so that the lower end of the conveyor was submerged in the extraction solvent and the upper end was above the solvent. The conveyor carries the solids forward such that the material was initially submerged in solvent and then the material emerged from the solvent and excess solvent drained back into the solvent stream. At the end of the conveyor, the solids dropped onto another conveyor with a similar arrangement. The model IV extractor had six extraction stages. Fresh solvent is introduced at the discharge end and flows towards the inlet end and is ultimately discharged at a point preceding the solids introduction. After the final solvent contact, the solids are conveyed up a long section to allow more extensive draining before falling into a crossover screw for transport to desolventizing. The solvent is fed into the system at 0.445 kg/min and the solids were introduced at 0.027 kg/min (based on a volumetric feeder) and the solvent is maintained at 59° C. by in situ heat exchangers. Total solvent to solids ratio is about 16 and total contact time is about 60 minutes. The water of the extraction system is introduced through a combination of carryover water from the input material and water in the fresh solvent. The composition of the feed solvent to contact the extracted destarched corn gluten is approximately 97.3% ethanol and 2.7% water. Consequently, the composition of the solvent varied across the extractor, but the final solvent concentration was about 97% ethanol. Desolventizing occurred in a Bepex Solidaire dryer operated with a surface temperature of about 155-160° C. and an absolute pressure from about 270-330 millibar (with a target of about 300 millibar).

The resulting corn protein concentrate product is about 57.5% protein (dry basis). Further, the oil is less than 0.5% on a dry basis, the product color, as measured using the Hunter colorimeter, has “L*” color equal to 89.9, “a*” color equal to 0.5 and “b*” color equal to 17.2. The free sulfite is 89 mg/kg (dry basis).

### Example 5: CPC Functionality in Processed Meat Products

The formation of a viscous dispersion or gel during and after heating can be useful in many food systems, often improving the texture or yield of the food. One possible non-limiting example of this functionality can be seen in a model system based on a beef frank, which is a kind of emulsified meat product. The model system was adapted from Paulson et al. (1984) Can. Inst. Food Sci. Technol. J. 17:202-208.

A 36 g sample of 93% lean ground beef is weighed into a dish and stored at about 4° C. until use. A 45 g sample of lard (Armour) is weighed into a separate dish and stored at ambient temperature (20-25° C.) until use. 25 g of cold tap water is weighed into a centrifuge tube and stored at 4° C. until use. Another 33 g of tap water is weighed into a cup and stored at 4° C. until use. Salt (4.5 g) is weighed into a small dish and protein additive (4 g) is weighed into another small dish. Both of the latter are stored at ambient (20-25° C.) temperature until use.

A Cuisinart mixing bowl is mounted onto the base (Cuisinart Little Pro Plus). The protein for the batch is added to a tube containing 25 g of water, shaken and left to hydrate at room temperature for 2-4 minutes. The pre-weighed meat is added to the Cuisinart bowl and pulsed 2-3 times to break up the chunks. The salt is added and pulsed a few times. The hydrated protein and remaining water are added to the bowl and pulsed 2-3 times. Finally, the lard is added to the bowl and pulsed 2-3 times. The Cuisinart is run with constant mixing for 1 minute, the sides are scraped down, and the mixer run another one minute. Two 30 g samples are removed and placed into 50 mL plastic centrifuge tubes with screw top closures. After vigorous tapping to settle the material, the tubes are centrifuged for 1 minute at 3000 g to force out entrained air. The tubes are placed into a 75° C. water bath for 35 minutes to cook. At the end of the heating, the tubes are removed from the bath, allowed to partially cool, and the liquid is decanted into pre-weighed aluminum dishes and weighed. The liquid lost is subtracted from the initial weight and used to calculate the mean yield. A reference is prepared in the same way but without the protein added to the 25 g of water. The protein ingredient provides a substantial yield boost to the finished product. Results are highlighted in Table 11.

TABLE 11

| Sample | Yield (%) |
|---|---|
| Reference (no treatment) | 34.6 |
| CPCP119 | 68.7 |
| CPCP120 | 54.2 |

1. A corn protein concentrate, comprising:
a) 55%-80% corn protein on a dry basis;
b) an a* color value between about 0 and 4, and a b* color value between about 15 and 35; and
c) less than about 2% oil on a dry basis.

2. The corn protein concentrate of claim 1 wherein the corn protein is between about 55% and 75% on a dry basis.

3. The corn protein concentrate of claim 1 further comprising an L* color value ranging from 70 to 90.

4. The corn protein concentrate of claim 1 wherein the a* color value ranges between about 0 and 2.

5. The corn protein concentrate of claim 1 wherein the oil is less than about 1% on a dry basis.

6. The corn protein concentrate of claim 1 further comprising 13% to 23% starch on a dry basis.

7. The corn protein concentrate of claim 1 further comprising 13% to 16% starch on a dry basis.

8. The corn protein concentrate of claim 1 having a gel strength ranging from 0.15 to 0.20 N.

9. The corn protein concentrate of claim 1 having an insoluble carbohydrate concentration ranging from 15 to 18 g/kg on a dry basis.

10. The corn protein concentrate of claim 1 having an organic acid concentration of about 3.0 g/kg or less on a dry basis.

11. The corn protein concentrate of claim 1 having a free sulfite concentrate of less than 100 ppm.

12. A method of producing a corn protein concentrate, comprising:
a) Providing a corn gluten meal;
b) Washing the corn gluten meal with a solvent comprising water and a water-miscible organic solvent to obtain a corn protein concentrate, comprising:
i. 55%-80% corn protein on a dry basis;
ii. an a* color value between about 0 and 4, and a b* color value between about 15 and 35; and
iii. less than about 2% oil on a dry basis.

13. The method of claim 12 wherein the water-miscible solvent is ethanol and the concentration of ethanol is between about 85% and 99.5% on a mass basis.

14. The method of claim 12 wherein the water-miscible solvent is isopropanol and the concentration of isopropanol is between about 85% and 99.5% on a mass basis.

15. The method of claim 12 wherein the corn protein is between about 55% and 75% on a dry basis.

16. The method of claim 12 wherein the corn protein concentrate has an L color value ranging from 70 to 90.

17. The method of claim 12 wherein the corn protein concentrate comprises less than about 1% oil on a dry basis.

18. The method of claim 12 wherein the corn protein concentrate comprises 13% to 23% starch on a dry basis.

19. The method of claim 12 wherein the corn protein concentrate comprises 13% to 16% starch on a dry basis.

20. The method of claim 12 wherein the corn protein concentrate has a gel strength ranging from 0.15 to 0.20 N.

21. The method of claim 12 wherein the corn protein concentrate has an insoluble carbohydrate concentration ranging from 15 to 18 g/kg on a dry basis.

22. The method of claim 12 wherein the corn protein concentrate has an organic acid concentration of about 3.0 g/kg or less on a dry basis.

23. The method of claim 12 wherein the corn protein concentrate has a free sulfite concentration of less than 100 ppm.
